(19) United States
(12) Patent Application Publication
(10) Pub. No.: US 2004/0243177 A1
(43) Pub. Date: Dec. 2, 2004

(54) Manual insertion tool for a cochlear implant

(76) Inventors: Martin Svehla, Botany (AU); Peter Gibson, South Coogee (AU)

Correspondence Address: JAGTIANI + GUTTAG, 10363-A DEMOCRACY LANE, FAIRFAX, VA, 22030, US

(21) Appl. No.: 10/825,367

(22) Filed: Apr. 16, 2004

(30) Foreign Application Priority Data
Apr. 17, 2003 (AU) 2003901869

(51) Int. Cl. A61B 17/28

(52) U.S. Cl. 606/210; 606/207

(57) ABSTRACT

A clasping tool for controlling an elongate tubular member comprising a tip constructed to support and limit lateral movement of the tubular member relative to the tip, and to permit longitudinal movement of the tubular member relative to the tip and an opposing tip constructed to retain the tubular member between the tips when the tips are brought together. Tips may have a flat region, half-tube region, forked region, looped region, or V-shaped region for grasping, holding, supporting and releasing the elongated tubular member. Each tip may have a similar region.

### CROSS-REFERENCE TO RELATED APPLICATIONS

This application claims the priority of the following co-pending Australian Patent Application No. 2003901869, entitled “Manual Insertion Tool For a Cochlear Implant,” filed on Apr. 17, 2003. The entire disclosure and contents of the above application are hereby incorporated by reference.

### BACKGROUND

1. Field of the Invention

The present invention generally relates to surgical tools and more particularly to surgical tools that may be used to grasp, hold, grip, release, etc. implantable and non-implantable medical devices and components of those devices.

2. Related Art

The use of implantable medical devices to apply therapy to the body is becoming increasingly common as the benefit that such devices provide become fully realized. Typically, such devices require the implantation and strategic placement of electrode arrays close to sensitive structures of the body to apply stimulation thereto, typically in the form of electrical or mechanical stimulation. Devices such as cardiac pacemakers, prosthetic hearing implants, such as Cochlear™ implants sold by Cochlear Limited, and implantable hearing aids are all typical examples.

Often, the procedure for implanting and locating the devices within the body requires much skill by the surgeon and the dexterous use of existing surgical tools to achieve the desired device placement. The implantable elements are also often of a size and shape that increases the difficulty of their handling, and the tools that are employed to handle such devices are generally not specifically designed to perform the given task.

### SUMMARY

According to one aspect of the present invention, there is provided a clasping tool for controlling an elongate tubular member comprising a tip constructed to support and limit lateral movement of the tubular member relative to the tip, and to permit longitudinal movement of the tubular member relative to the tip and an opposing tip constructed to retain the tubular member between the tips when the tips are brought together.

According to another aspect of the invention, there is provided a tool for a clasping tool for controlling an elongate tubular member comprising a first tip means for supporting and limiting lateral movement of the tubular member relative to the first tip means, while permitting longitudinal movement of the tubular member relative to the first tip means, and a second tip means for retaining said tubular member between said first and second tips when said first and second tips are brought together.

### BRIEF DESCRIPTION OF THE DRAWINGS

The invention will be described in conjunction with the accompanying drawings, in which:

FIG. 1 is a view of the implanted components of a typical hearing implant system;

FIG. 2 is a simplified view of an electrode array assembly of the implant system of FIG. 1;

FIG. 3A is a perspective view of an embodiment of the tool of the present invention;

FIG. 3B is a side view of the embodiment shown in FIG. 3A;

FIG. 3C is a top view of the embodiment shown in FIG. 3B;

FIG. 4A is a perspective view of an embodiment of the tool of the present invention having one tip with a half-tube region and an opposing tip with a flat region;

FIG. 4B is a detailed perspective view of the flat region of a tip shown in FIG. 4A;

FIG. 4C is a detailed perspective view of the half-tube region of a tip shown in FIG. 4B;

FIG. 5A is a top view of an half-tube region of a tip of the present invention;

FIG. 5B is a end view of the half-tube region of FIG. 5A;

FIG. 5C is a side view of the half-tube region of FIG. 5A;

FIG. 6A is a top view of an half-tube region having an aperture of a tip of the present invention;

FIG. 6B is a end view of the half-tube region of FIG. 6A;

FIG. 6C is a perspective view of the half-tube region of FIG. 6A;

FIG. 7A is a side view of half-tube region that smoothly transitions into arm of a tool constructed in accordance with an embodiment of the present invention;

FIG. 7B is a perspective view of the half-tube region shown in FIG. 7A;

FIG. 8A shows two interoperate tips of an embodiment of the present invention, with one tip having a looped region and the opposing tip having a flat region;

FIG. 8B shows two interoperate tips of an embodiment of the present invention, with one tip having a forked region and the opposing tip having a flat region;

FIG. 9 is a cross-section end view of an embodiment of the tip arrangements of the present invention that have a forked region and a flat region;

FIG. 10 represents various embodiments of a flat region of the present invention;

FIG. 11 shows two interoperate tips of an embodiment of the present invention where both tips have substantially similar V-shape regions;

FIG. 12 is an end view of the tip shown in FIG. 11; and

FIG. 13 is a side view of the tip show in FIG. 11.

### DETAILED DESCRIPTION

An exemplary embodiment present invention provides a tool that may be designed to grasp or hold an elongated tubular member or rod-shaped element, such as a tube, and allow easy manipulation of the element in a microenvironment, such as surgery. Embodiments of the present invention may be used to grasp any type of implantable or non-implantable medical elements, such as guidewires, catheters, or other elements elongated tubular portion.

The present invention will be described for use in the implantation of a prosthetic hearing implant, such as a Cochlear™ implant sold by Cochlear Limited. It should be understood to those skilled in the arts that the present invention may be used in other surgical procedures which require grasping or holding an elongated tubular member.

In terms of prosthetic hearing implants, electrical stimulation of the cochlea using the implant systems may be used to directly deliver electrical stimulation to the auditory nerve fibers, thereby allowing the brain to perceive a hearing sensation resembling the natural hearing sensation normally delivered to the auditory nerve. Surgical procedures required to insert a implant requires skill and precision due to the small area in which to operate and the delicate nature of the ear, especially in children.

Such implant systems have typically consisted of two main components, an external component commonly referred to as a processor unit and an internal implanted component commonly referred to as a receiver/stimulator unit. Traditionally, both of these components have cooperated together to provide the sound sensation to a recipient.

The external component has traditionally consisted of a microphone for detecting sounds, such as speech and environmental sounds, a speech processor that converts the detected sounds, particularly speech, into a coded signal, a power source such as a battery, and an external transmitter antenna. The coded signal output by the speech processor is transmitted transcutaneously to the implanted receiver/stimulator unit situated within a recess of the temporal bone of the recipient. This transcutaneous transmission occurs via the external transmitter antenna which is positioned to communicate with an implanted receiver antenna provided with the receiver/stimulator unit. This communication serves two essential purposes, firstly to transcutaneously transmit the coded sound signal and secondly to provide power to the implanted receiver/stimulator unit. Conventionally, this link has been in the form of a radio frequency (RF) link, but other such links have been proposed and implemented with varying degrees of success.

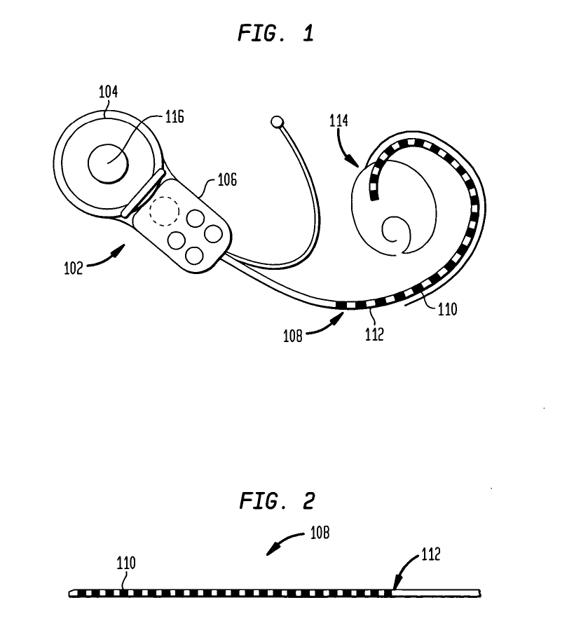

FIG. 1 shows an implanted unit 102 having a receiver/transmitter antenna coil 104 that receives the coded signal and power from the external processor component (not shown), and a stimulator 106 that processes the coded signal and outputs a stimulation signal to an intracochlear electrode assembly 108. Electrode assembly 108 has several electrodes 110 in an electrode array that are mounted in an electrode carrier member 112 which applies the electrical stimulation directly to the auditory nerve producing a hearing sensation corresponding to the original detected sound. Electrode assembly 108 is typically implanted in the scala tympani of cochlea 114. In operation, unit 102 contains the relative electrical circuitry to convert coded signals received from the external speech processor (not shown) into stimulation pulses to be applied by a selected electrode 110 strategically placed within cochlea 114. In this regard, receiver antenna coil 104 receives the coded signal from an external transmitter antenna coil (not shown) aligned therewith via a transcutaneous radio frequency (RF) link. Alignment of the external transmitter antenna coil with receiver antenna coil 104 is typically achieved by a magnet 116 placed centrally of receiver antenna coil 104 and a magnet (not shown) in the external transmitter antenna coil which magnetically holds both coils in place for transmission to occur.

As is shown in FIG. 2, intracochlear electrode assembly 108 typically consists of a plurality of electrodes 110, encapsulated in an electrode carrier member 112. Electrode carrier member may be made from a flexible material, such as silicone. Each electrode 110 may be connected by at least one conductor element or wire (not shown), electrically connecting the electrode element 110 to unit 102. In this regard, electrode assembly 108 may be a flexible, substantially tubular configuration having a substantially oval or circular cross-section. In a preferred form, electrode assembly 108 is preferably constructed in a pre-curved configuration to conform to the natural spiral shape of the cochlea, and held in a straight configuration for insertion through the use of a stiffening stylet (not shown) which extends substantially the length of the electrode assembly 108.

In addition, an electrode assembly may be in a carrier member, such as those described and shown in Australian Provisional Patent No. 2003901852, entitled “Cochlear Electrode Array,” filed on Apr. 16, 2003, and U.S. Patent entitled “Cochlear Electrode Array,” filed on concurrently with the present invention. The entire contains and disclosures of these applications are hereby incorporated by reference.

The electrode assembly may be placed within a duct of the cochlea referred to as the scala tympani. The procedure for performing this task and ensuring that the assembly is correctly positioned within the cochlea is a relatively difficult one requiring much skill and care on behalf of the surgeon. To position the electrode assembly, a surgeon must first access the cochlea and this is typically done by performing a mastoidectomy and posterior tympanotomy, followed by a cochleostomy to create an opening to the cochlea with which the electrode assembly is to be inserted. The electrode assembly may be inserted by gripping the electrode carrier member by hand or with a tool and inserting a leading end of the electrode carrier member through the cochleostomy and into preferably the scala tympani of the cochlea.

Conventional tools are not suited to insert the electrode carrier member in a manner that does not risk damage to the ear. Since there is the possibility of potential of irreversible damage to the cochlea, an advantage provided by the present invention may be to enable one to grasp, hold, retain and release the elongated insert element, i.e. electrode carrier member, in a controlled and stable manner during insertion. One advantage of an embodiment of the present invention may be that the clasping tips of the tool provide the ability of a surgeon to grasp, hold, retain and release an electrode carrier member without reducing maneuverability and/or visibility through in tight spatial constraints of the surgical sites, such as spatial constraints of the posterior tympanotomy. In addition, an advantage of an embodiment of the present invention may be to allow a minimal movement of the tool when releasing the insert element with one tip, while maintaining support with the other tip. The tip that is released moves to extend slightly above the elongated insert member such that the tip does not interfere with the insertion of the insert member.

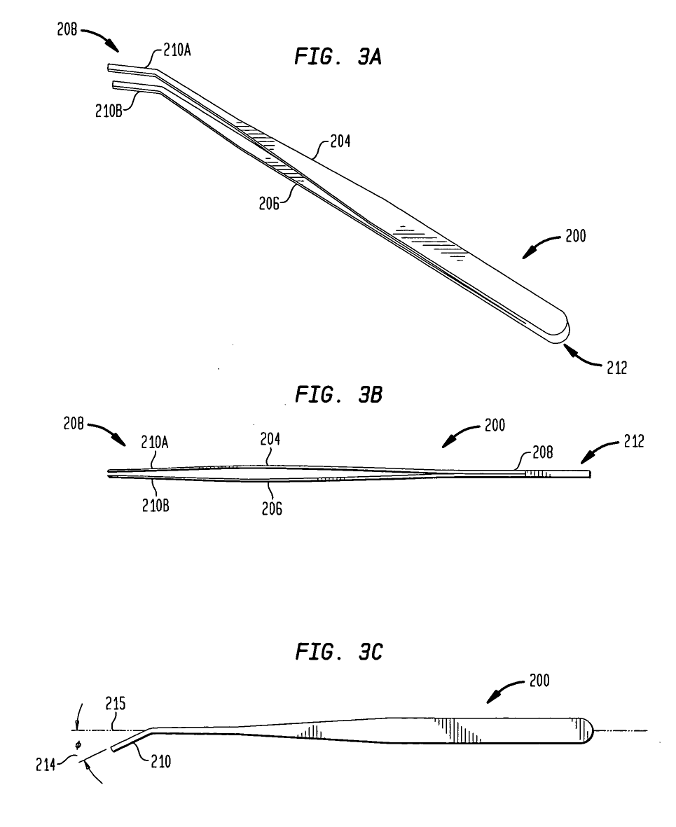

FIG. 3A is a perspective view and FIGS. 3B and 3C are side and top views, respectively, of an exemplary embodiment of a tool according to the present invention. A tool 200 has a body 202 which branches into two relatively flexibly movable arms 204 and 206. Arms 204 and 206 terminate at end 208 in respective tips 210A, 210B which are adapted to hold or capture a substantially tubular element, such as electrode assembly 108, therebetween. Arms 204 and 206 are fixed together with respect to each other at one end 212, and are biased such that tips 210 of arms 204 and 206, when in a relaxed position, are positioned remote from each other at end 208. In this regard, tips 210 may be brought together by applying a compressive force on movable arms 204 and 206 to hold or capture an element between tips 210.

As is evident in FIGS. 3A and 3C, tips 210 are positioned at an angle Φ 214 offset from the longitudinal axis 215 of tool 200. In one embodiment, angle 214 may be any angle from approximately 0° to 25°, more preferably approximately 18° to 20°. In applications for inserting an electrode carrier member into the cochlea, angle 214 that is greater than 25° may reduce the directional capability of tool 200. However, it should be understood to those skilled in the art that angle 214 may be greater than 25° for different applications. An offset angle may allow relative longitudinal movement of an electrode carrier member in which the tool does not interfere when one tip is released and the electrode carrier member is inserted into the cochlea.

As is further evident from these figures, tips 210 are provided with an elongated, constant small diameter section that differs significantly from standard forceps/tweezers, which typically gradually increase in cross section from the tip back to the main body of the tool. The length of the offset tips 210 may vary depending upon the use of the tool, but the length may be preferably between approximately 5 to 15 mm, more preferably about 9 mm. In one embodiment, the entire length of tool 200 from end 208 to end 212 may be approximately 130 to 200 mm, more preferably approximately 145 to 160 mm. It should be understood that for different embodiments, the entire length of tool 200 may vary.

One purpose of the angled and elongated, constant diameter section of the tips 210 may be to allow improved access and manipulation of the tool and any tubular element held by the tool. In particular, a purpose of the region of the tips allows the tool to be used in areas where there is restricted access for conventional surgical tools, such as to gain access through the posterior tympanotomy in an implantation procedure. Angled tips provide improved visibility of the site to the surgeon whilst being substantially straight to allow a precise “dart like” manipulation of the tool.

A tool may be from a metal such as 304 Stainless steel, however the tool could also be made from a ceramic material using a material such as alumina. Such a device would allow re-usability of the tool following surgical use and sterilisation. However, it is also envisaged that the tool may be disposable following use, and in this regard the tool may be made of a plastic, such as PEEK, ABS, PMMA, Polyimide, etc. In addition, tips may have a surface finish that provides an increased in gripping ability of the tool.

In an embodiment of the present invention, the arms of a tool of the present invention may be designed to allow a surgeon or other user to grip by hand to cause the arms to move towards each other when compressed and move away from each other when released. A portion of arm may have a greater width to accommodate a finger or thumb. At the widest portion, arms may have a width of approximately 10 mm.

In an embodiment of the present invention, there is provide a post located on arm 206 and that extends from arm 206 towards arm 204. When tool 200 is in a relaxed position, there may be a gap between the post and arm 204. When tool 200 is compressed, post may prevent a tip on arm 204 from contacting a tip on arm 206. This design may be used to prevent a surgeon from inadvertently applying an excessive compressive force or squashing the insert member, i.e., electrode carrier member, when applying force to the arms.

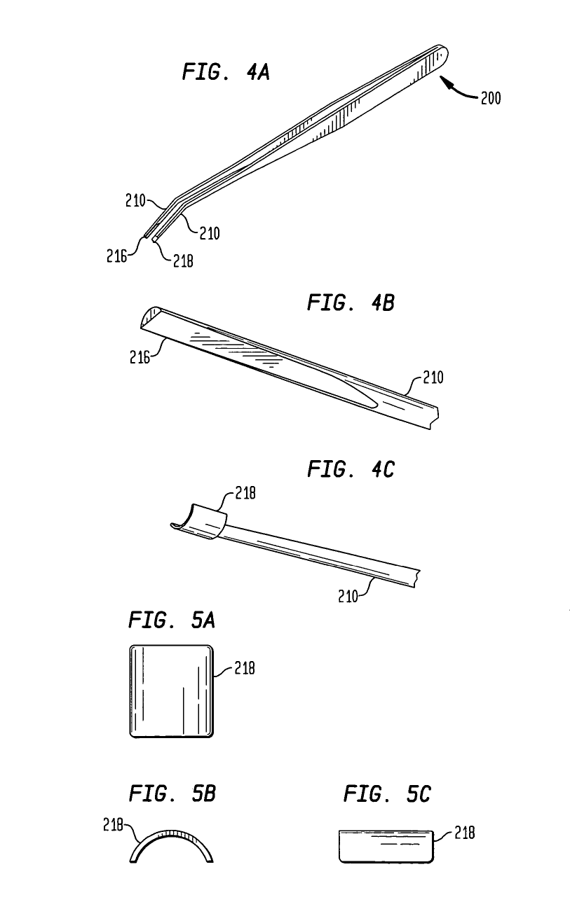

FIG. 4A is a perspective view of one embodiment of tool 200 according to the present invention. FIGS. 4B and 4C show in more detail the angled and elongated, constant small diameter section of tips 210 of the present invention according to one embodiment. As shown, tips 210 are designed to cooperate together to securely and safely hold and maintain in control a substantially tubular element therebetween, such as an electrode assembly. In this particular embodiment, one of the tips 210 may be provided with a substantially flat face 216, with the other tip provided with a substantially half-tube region 218.

Half-tube region 218 may be substantially semi-circular or having a “U” shaped cross section that provides an increase contact area with electrode carrier member and may limits relative lateral movement of the electrode carrier member. As discussed below, other tip shapes may perform a similar support function, such as forks, loops, “V” shapes, etc.

This embodiment differs substantially from presently used surgical forceps or tweezers where the tips are substantially sharp, flat ends. When such tools are employed to grip an element, such as a substantially tubular electrode assembly 108 shown in FIG. 2, the contact area between the electrode assembly and the forcep tips is quite small and does not sufficiently constrain the electrode assembly between the ends of the tool, resulting in the array easily swinging between the two tips.

In the embodiment of the present invention shown in FIGS. 4A and 4C, half-tube region 218 provides a larger contact area to more firmly constrain or support the electrode carrier member of an electrode assembly therebetween, with the holding or grasping force being applied by flat face 216 of opposing tip 210 of tool 200. This may provide an improved surgical control by limiting lateral movement of the electrode member relative to tip 210 when one tip is released after orientating the electrode assembly during the insertion procedure, thereby providing a procedure where potential damage to the sensitive structure of the cochlea is relatively minimized, as is damage to the structure of the electrode array, through mishandling.

As mentioned, flat face 216 of the tip 210 aids in constraining the tubular element within half-tube region 218 of the other tip 210. As in the case of prosthetic hearing implants, there is often a very confined spatial area within which the tool must operate and a flat face region allows for easy release of the electrode element in the confined space, then would be the case should both tips be of a half-tube configuration. It has been found that with such a configuration shown in FIG. 4A that the arms of the tool may be easily opened, even in instances where the space for doing so is limited, such as within a posterior tympanotomy. The flat face region may be wider than the outer diameter of the opposed half-tube region. This may ensure that when a tool is used to manipulate an electrode assembly that is thinner than the half-tube region that the tool will not squash or apply a damaging force to the electrode assembly when there is force placed upon the arms of the tool by the surgeon. A flat face region that is narrower than the outer diameter of the opposed half-tube region may provide a surgeon more control on the force imparted on the electrode carrier member.

FIGS. 5A, 5B and 5C are a top view, end view and side view, respectively, of half-tube region 218 constructed in accordance with one embodiment of the present invention shown in FIG. 4A. As may be appreciated, the geometry of the half-tube region may be altered to optimize the holding capabilities of the tool, without adversely affecting the visibility of the user of the tool. In one embodiment, the thickness of the half-tube region approximately 0.1 to 0.2 mm. Depending on the materials used and implemented in construction tools having half-tube regions thinner than approximately 0.1 mm may suffer from reduced tip strength resulting in an inability of the tool to firmly grasp the tubular element, and with thicker regions, i.e., greater than approximately 0.3 mm, may adversely affect the visibility of the user when manipulating the device.

In one embodiment the length of half-tube region may be approximately 0.8-1.2 mm, which may be long enough to stably hold a tubular element such as an electrode array, and yet short enough to maintain flexibility of use of the tool. Typically, half-tube region 218 may be designed to subtends an arc of less than or equal to approximately 180°. Any greater than this and the arc may cause difficulties placing the electrode assembly into the tool.

In one embodiment the width of half-tube region may be approximately equal or slightly less than the diameter of insert member, i.e. electrode carrier member. A width may be greater than or significantly less than the diameter the electrode carrier member may result in a non-firm grip or hold.

Whilst the embodiments shown in FIG. 4B represent semi-circular shapes, it is envisaged that the half-tubular regions could equally be made of a square, semi-hexagon or apex of a triangle, to receive the tubular member therein. Additional variants of shape may be used that have outer sides that extend further towards to the opposing tip than the middle.

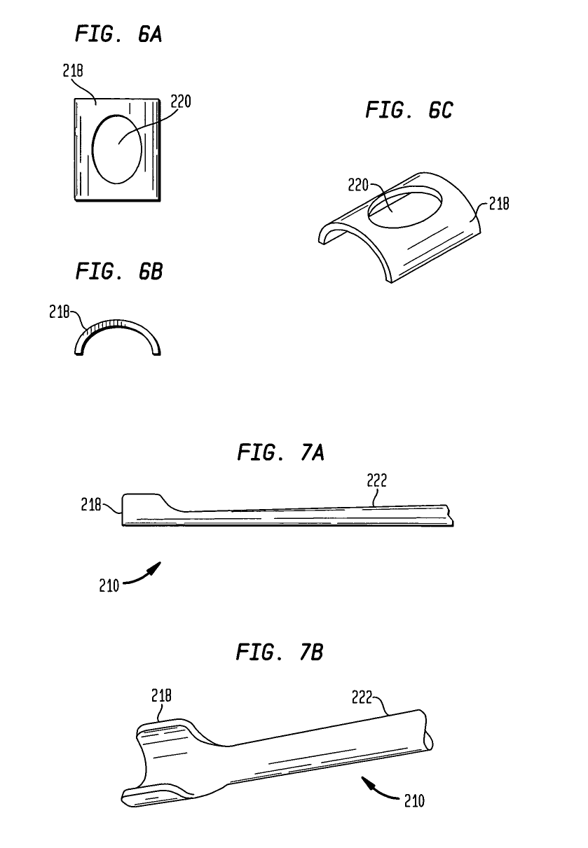

FIGS. 6A, 6B, and 6C are a top view, end view and side view, respectively, showing an alternative embodiment of half-tube region 218 of FIG. 4A. In this embodiment, a cut out section or aperture 220 is provided in half-tube region 218 to aid in the user's visibility of the tip of the tool. In one embodiment, aperture 220 may be placed in a central location of half-tube region 218.

FIG. 7A is a side view and FIG. 7B is a perceptive view of an alternative embodiment of the present invention adapted to provide a gradual conversion from the straight section 222 of arms 204 or 206 to half-tube region 218. In this embodiment all sharp angles of the tool are minimized, and as such the risk of the catching the tool on the electrode assembly during removal of the insert member from the tool may be reduced.

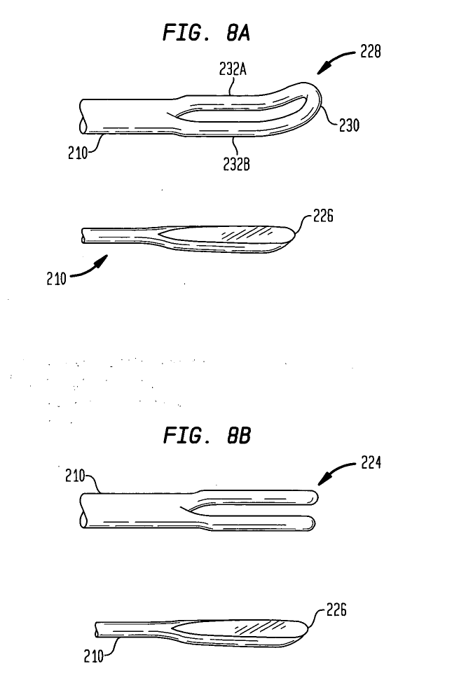

FIG. 8A is side view showing an embodiment of the configuration of tips 210 of tool 200 of the present invention. In this embodiment, tips 210 are still designed to firmly maintain a tubular element therebetween, however instead of a half-tube region receiving the tubular element, looped region 228 is employed to act with a flat region 226 of opposed tip 210. Looped region has an end 230 that bends away from flat region 226 and fork elements 232A, 232B that form an opening. Electrode array 108 will be securely held between the looped region 228 by fork elements 232A, 232B and a flat region 226 of opposing tip 210, in a similar manner as is achieved with a half-tube design shown in FIG. 4A.

FIG. 8B is side view showing an embodiment of the configuration of tips 210 of tool 200 of the present invention. In this embodiment, tips 210 are still designed to firmly maintain a tubular element therebetween, however instead of a half-tube region receiving the tubular element, forked region 224 is employed to act with a flat region 226 of opposed tip 210. Forked region 224 has an open end. Electrode array 108 will be securely held between the looped region 228 by fork elements 232A, 232B and a flat region 226 of opposing tip 210, in a similar manner as is achieved with a half-tube design shown in FIG. 4A.

Although FIG. 8B shows forked region 224 having a substantially constant distance from flat region 226, the end of forked region 224 may bend away from flat region as shown in FIG. 8A. Likewise, looped region 228, shown in FIG. 8A may have a substantially constant distance from flat region 226 as shown in FIG. 8B.

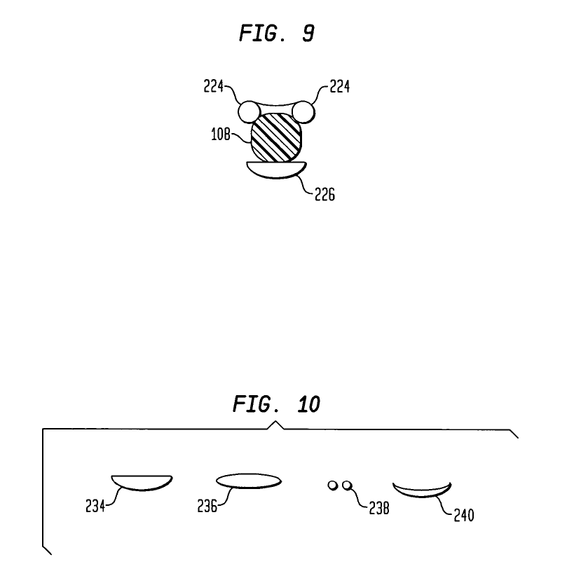

FIG. 9 is a cut-away end view showing the present embodiment grasping an electrode assembly 108 using forked region 224 and a flat region 226. For example, a compressive force may be applied to arms causing forked region 224 grasp electrode carrier member 108 in a similar fashion as the half-tube region shown in FIG. 4C. Once the compressive force is released, forked region 224 may release electrode carrier member 108.

Although a tool is shown with one tip having a forked region or looped region and the opposing tip having a flat region, both tips may have a forked and/or looped region. In addition, in embodiment where both tips are forked or looped regions, the ends may bend away has shown in FIG. 8A or have a substantially constant distance as shown in FIG. 8B.

One additional feature in a forked or looped region having an opened end may be insert one or more crossbeams between the fork elements to help stabilize the fork elements at a point along the length of the loop. The position of a crossbeam may be determined by the optimal position that would least impact on the view of the electrode carrier member. For example, if the position of the crossbeam was out of the region of the electrode carrier member being held the beam could be in a plane of the loop, otherwise crossbeam would preferably be an arc-type crossbeam to prevent contact of electrode carrier with the cross beam prior to the fork elements contacting the electrode carrier member.

In each of the above described embodiments, the tips are generally formed with one shaped tip capable of receiving a tubular element therein and an opposed flat tip acting together with the shaped tip to retain or hold the tubular element securely therebetween. It should be appreciated that a variety of shapes, other than flat, could be used on the opposing tip and still remain within the spirit of the present invention.

FIG. 10 provides cross-sectional end views of embodiments of a tip in accordance with embodiment of the present invention, providing a flat 234, convex 236, forked/looped 238 or concave 240 region.

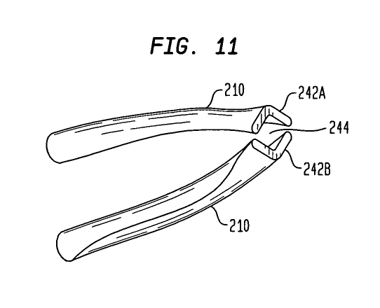

FIG. 11 shows an embodiment of the present invention wherein both tips 210 are configured with a substantially identical concave arrangement. As shown in FIG. 11, tip 210 has tip end region 242A, 242B which define a region 244 where a tubular element such as an electrode assembly, may be maintained securely by tip end region 242.

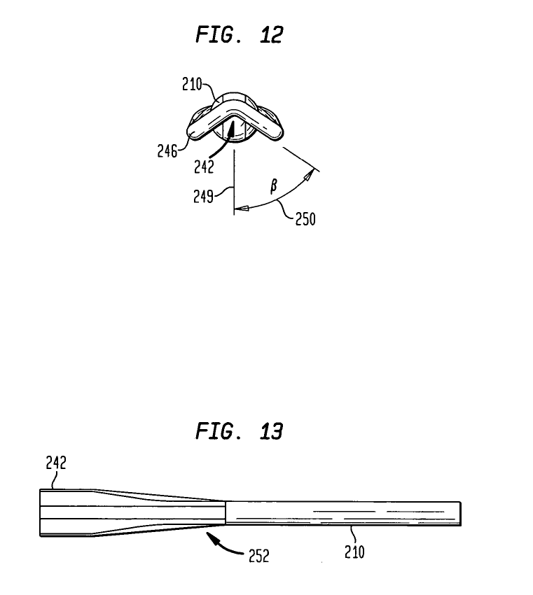

FIG. 12 shows an end view of a tip end region 242 constructed in accordance with one embodiment of the present invention. Tip 210 has a substantially curved V-shaped grooved end region 242 rather than a half-tube design, to optimise grip between the two acting tips when brought together about a tubular member. End region 242 may have a thickness of approximately 0.15 mm. In addition, each side 246 of end region 242 may be offset from the vertical axis 249 by an angle β 250 of approximately 55° degrees.

FIG. 13 shows a side view of a tip 210 having V-shaped groove end region 242. Tip 210 may be offset from the arm of the tool, as shown in FIG. 3A, before tampered portion 252 that extends toward V-shaped groove end region 242. Tip 210 may have a length of approximately 5 mm from the offset of the arm to tampered portion 252 and a thickness of approximately 0.5 mm. Tampered portion 252 may have a length of approximately 3 mm from tip 210 toward the V-shaped groove end region 242. V-shaped groove end region 242 may have a length of 1 mm and a width of approximately 0.9 to 1.5 mm, preferably, 1.02 mm.

As may be appreciated, the present invention provides a surgical tool capable of performing delicate manual microsurgical procedures with stability and control. This is achieved by providing an angled, elongated, substantially constant small diameter section near the tips of the tools to be manipulated to securely maintain a tubular member therebetween for manipulation during surgery. Unlike more conventional surgical tools, the present invention provides tip regions specifically designed to receive tubular elements such as electrode arrays and to enable handling of such elements in both a secure and stable manner as well as in a manner that will not damage the delicate structures of the elements.

As discussed above, embodiments of the present invention provides a surgical tool capable of performing delicate manual microsurgical procedures with stability and control. Unlike more conventional surgical tools, the present invention provides clasping tip regions specifically designed to receive tubular elements such as electrode arrays and to enable handling of such elements in both a secure and stable manner as well as in a manner that may not damage the delicate structures of the elements.

Whilst the present invention was described in relation to a forcep-like tool, the present invention is equally applicable to a pincer-type tool or alligator clamp tool that may be used for grasping elements in a surgical situation. It should be appreciated that the present invention is not limited to the hand-interface aspect of the tool.

All documents, patents, journal articles and other materials cited in the present application are hereby incorporated by reference.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart therefrom.

What is claimed is:

1. A clasping tool for controlling an elongate tubular member comprising:
a first tip constructed to support and limit lateral movement of said tubular member relative to said first tip, and to permit longitudinal movement of said tubular member relative to said first tip; and
a second tip constructed to retain said tubular member between said first and second tips when said first and second tips are brought together.

2. The tool of claim 1, wherein said tubular member is an electrode carrier member.

3. The tool of claim 2, wherein said electrode carrier member is connected to a stimulating unit of a prosthetic hearing implant device.

4. The tool of claim 1, wherein said first and second tips are connected to a first movable arm and second movable arm, respectively, which are joined at an end opposing said first and second tips, wherein application of a compressive force to said first and second movable arms to cause said first and second tips to be brought together.

5. The tool of claim 4, wherein said first and second tips are offset from a longitudinal axis formed by said first and second movable arms by an angle of approximately 0° to 25° degrees.

6. The tool of claim 5, wherein said offset angle is approximately 18° degrees.

7. The tool of claim 1, wherein said first tip has a half-tube region having a pair of opposing sides that extend toward said second tip.

8. The tool of claim 7, wherein said half-tube region has a thickness of approximately 0.1 mm.

9. The tool of claim 7, wherein said half-tube region has an aperture.

10. The tool of claim 1, wherein said first tip has a forked region.

11. The tool of claim 10, wherein said forked region has a pair of forked elements that are curved away from said second tip at the end of said forked region.

12. The tool of claim 1, wherein said first tip has a looped region having two fork elements and an aperture.

13. The tool of claim 12, wherein said looped region has a closed end that is curved away from said second tip.

14. The tool of claim 1, wherein said second tip has a substantially flat region facing toward said first tip.

15. The tool of claim 14, wherein said substantially flat region extends slightly wider than the width of said first tip and contacts said first tip when said first and second tips are brought together.

16. The tool of claim 14, wherein said substantially flat region extends slightly narrower than the width of said first tip and contacts said tubular member when said first and second tips are brought together.

17. The tool of claim 1, wherein said first and second tips have the same region.

18. The tool of claim 1, wherein said first and second tips have a substantially V-shaped region that forms a holding region for said tubular member when said first and second tips are brought together.

19. A clasping tool for controlling an elongate tubular member comprising:
a first tip means for supporting and limiting lateral movement of said tubular member relative to said first tip means, while permitting longitudinal movement of said tubular member relative to said first tip means; and
a second tip means for retaining said tubular member between said first and second means tips when said first and second means tips are brought together.
